(19) United States
(12) Patent Application Publication
(10) Pub. No.: US 2023/0307100 A1
(43) Pub. Date: Sep. 28, 2023

(54) INFORMATION PROCESSING APPARATUS, INFORMATION PROCESSING SYSTEM, AND INFORMATION PROCESSING METHOD

(71) Applicant: Yuuki OHTAKA, Kanagawa (JP)

(72) Inventor: Yuuki OHTAKA, Kanagawa (JP)

(73) Assignee: Ricoh Company, Ltd., Tokyo (JP)

(21) Appl. No.: 18/181,081

(22) Filed: Mar. 9, 2023

(30) Foreign Application Priority Data
Mar. 22, 2022 (JP) 2022-045719

(51) Int. Cl. G16H 10/60; G16H 15/00

(52) U.S. Cl. CPC G16H 10/60; G16H 15/00

(57) ABSTRACT

An information processing apparatus includes a memory and circuitry. The circuitry is configured to connect to an external service that manages a user's activity history, acquire first data related to the user's activity history from the external service, and receive second data related to the user's health that is measured by a biometric sensor. The circuitry is configured to store, in the memory, aggregation setting information in which information used for aggregation of the first data and the second data is set for one or more aggregation items, for each of which the first data and the second data are aggregated and displayed. The circuitry is configured to generate aggregated data corresponding to at least one aggregation item selected by the user. The circuitry is configured to output the generated aggregated data corresponding to the at least one aggregation item selected by the user.

### CROSS-REFERENCE TO RELATED APPLICATIONS

The present application claims priority under 35 U.S.C. § 119(a) to Japanese Patent Application No. 2022-045719, filed Mar. 22, 2022 in the Japan Patent Office, the contents of which are incorporated herein by reference in their entirety.

### BACKGROUND OF THE INVENTION

### 1. Field of the Invention

Embodiments of the present disclosure relate to information processing devices, information processing systems, and information processing methods.

### 2. Description of the Related Art

It has become common, for example, for companies to use external services such as groupware to perform organizational management and attendance management and to provide operational support to users such as employees. Furthermore, it has also become common to acquire biometric information from users wearing biometric sensors.

In Patent Document 1, a stress value is calculated from the biometric information of a person being managed, and stressors are analyzed based on multiple pieces of external information corresponding to the date and time at which the stress value reached a predetermined value or more.

However, in the configuration disclosed in Patent Document 1, it is only disclosed that the result of the analysis of the stress value performed based on the multiple pieces of external information is provided to the user. Thus, the configuration disclosed in Patent Document 1 cannot provide the user with a desired aggregated result of the information acquired from the biometric sensor and the multiple pieces of external information.

### RELATED-ART DOCUMENTS

### Patent Document

[Patent Document 1] Japanese Unexamined Patent Application Publication No. 2021-146053

### SUMMARY OF THE INVENTION

According to an embodiment, an information processing apparatus includes a memory and circuitry. The circuitry is configured to connect to an external service that manages activity history of a user, to acquire first data related to the activity history of the user from the external service, and to receive second data related to the user's health that is measured by a biometric sensor. The circuitry is also configured to store, in the memory, aggregation setting information in which information used for aggregation of the first data and the second data is set for one or more aggregation items, for each of which first data and the second data are aggregated and displayed. The circuitry is also configured to generate, based on the acquired first data, the received second data, and the stored aggregation setting information, aggregated data corresponding to at least one aggregation item that is selected by the user from the one or more aggregation items. The circuitry is further configured to output the generated aggregated data corresponding to the at least one aggregation item that is selected by the user.

### BRIEF DESCRIPTION OF THE DRAWINGS

FIG. 1 is a diagram illustrating an example of the configuration of an information processing system according to the embodiment;

FIG. 2 is a block diagram illustrating an example of the hardware configuration of a computer according to the embodiment;

FIG. 3 is a block diagram illustrating an example of the functional configuration of the information processing system according to the embodiment;

FIG. 4 is a view illustrating an example of the configuration of a chart type table;

FIG. 5 is a flowchart illustrating an example of data acquisition processing;

FIG. 6 is a flowchart illustrating an example of data output processing;

FIG. 7 is a view illustrating an example of a UI screen including charts;

FIG. 8 is a view illustrating another example of the UI screen including a chart;

FIG. 9 is a diagram illustrating an example of the configuration of the information processing system according to the embodiment; and

FIG. 10 is a diagram illustrating an example of the information processing system according to the embodiment.

### DESCRIPTION OF THE EMBODIMENTS

According to the disclosure, aggregated data desired by the user can be provided based on data acquired from external services and biometric sensors.

Embodiments will be described below with reference to the accompanying drawings.

[First Embodiment]

<System Configuration>

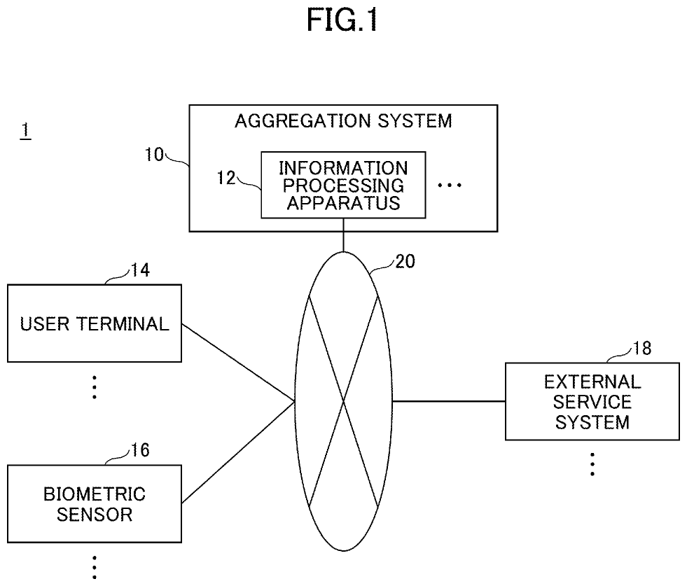

FIG. 1 is a diagram illustrating an example of the configuration of an information processing system according to the embodiment. An information processing system 1 illustrated in FIG. 1 includes an aggregation system 10, one or more user terminals 14, one or more biometric sensors 16, and one or more external service systems 18. The aggregation system 10 includes one or more information processing apparatuses 12. The information processing apparatuses 12, the user terminals 14, the biometric sensors 16, and the external service systems 18 communicate via a network 20. The network 20 is, for example, the Internet and/or a local area network (LAN).

The information processing apparatus 12 acquires data related to a user's activity history from the external service system 18 and receives data related to the user's health from the biometric sensor 16. The information processing apparatus 12 executes processing to aggregate the received data related to the user's activity history and the acquired data related to the user's health. The information processing apparatus 12 executes processing to cause the user terminal 14 to display the aggregated data based on the received data related to the user's activity history and the acquired data related to the user's health. The data (first data) related to the user's activity history acquired from the external service system 18 are data that are stored in the external service system 18 based on the user's use of an external service. Examples of external services include, for example, services related to an organizational management service, services related to attendance management, and services related to operational support. An external service may be a service related to groupware that has functions for emailing, chatting, scheduling, meeting management, attendance management, organizational management, and the like.

The data (second data) related to the user's health received from the biometric sensor 16 are data (biometric data) that are measured by the biometric sensor 16. The biometric data are, for example, measured by the biometric sensor 16 that may be a wearable device worn by the user.

The information processing apparatus 12 is, for example, a personal computer (PC) or a work station. The information processing apparatus 12 may be implemented by a server apparatus, an application service provider (ASP), or cloud computing.

The user terminal 14 is an information processing terminal operated by a user. The user of the terminal 14 may be, for example, an employee of an organization such as a company using the external service. The user may also be, for example, a manager managing the employee or a manager managing a system at an organization such as a company. The user terminal 14 may be, for example, a personal computer (PC), a cell phone, a smartphone, a tablet terminal, a gaming device, or a personal digital assistant (PDA).

The user terminal 14 displays a user interface (UI) such as a Web user interface provided by the aggregation system 10. The user operating the user terminal 14 configures the data acquisition settings and the data aggregation settings (both of which are to be described later) on the UI provided by the aggregation system 10. Further, the user operating the user terminal 14 can display the aggregated data, which are aggregated by the aggregation system 10 in accordance with the data acquisition settings and the data aggregation settings, on the UI provided by the aggregation system 10.

The biometric sensor 16 is, for example, worn by a user whose biometric data are to be measured. The biometric sensor 16 may be incorporated in a wearable device worn by the user whose biometric data are to be measured. The biometric sensor 16 may also be an Internet of Things (IoT) device. Furthermore, the biometric data received from the biometric sensor 16 may be data measured by the biometric sensor 16 or may be data obtained by using the measured data. The biometric data includes, for example, stress levels, average burned calories, and the like.

Each external service system 18 provides an external service to users of the information processing system 1. The external service system 18 also stores the data related to the activity history of each user who is using the external service. The data related to a user's activity history that are acquired from the external service are the data related to the user's activity history that are stored in the external service system 18.

Note that the configuration of the information processing system 1 illustrated in FIG. 1 is merely an example. For example, at least some of the functions of the information processing apparatus 12 may be provided in a device other than the information processing apparatus 12. For example, some of the functions of the information processing apparatus 12 may be provided in the user terminal 14, the biometric sensor 16, or the external service system 18. Further, the aggregation system 10 may be provided as a cloud-based service. Furthermore, the functions of the information processing apparatus 12 may be distributed among a plurality of computers.

<Hardware Configuration>

<<Computer>>

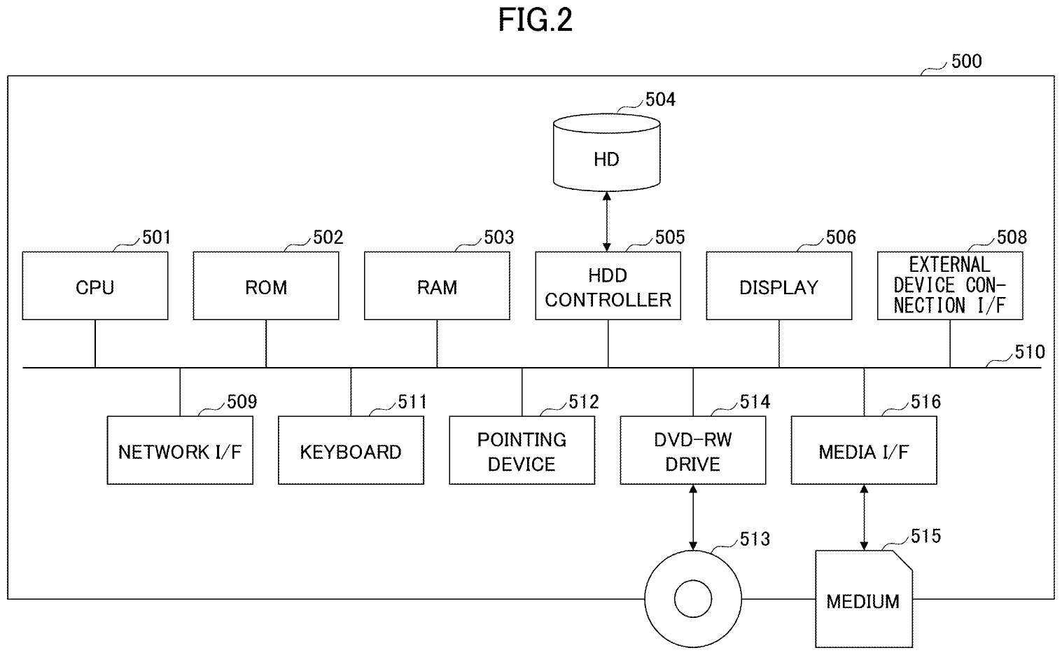

The information processing apparatus 12 and the external service system 18 illustrated in FIG. 1 each are implemented by, for example, a computer 500 having the hardware configuration as illustrated in FIG. 2. In a case where the user terminal 14 of FIG. 1 is to be implemented using a PC, it may be implemented by, for example, the computer 500 having the hardware configuration as illustrated in FIG. 2. FIG. 2 is a block diagram illustrating an example of the hardware configuration of a computer according to the embodiment.

The computer 500 includes a central processing unit (CPU) 501, a read only memory (ROM) 502, a random access memory (RAM) 503, a hard disk (HD) 504, a hard disk drive (HDD) controller 505, a display 506, an external device connection interface (I/F) 508, a data bus 510, a keyboard 511, a pointing device 512, a digital versatile disk rewritable (DVD-RW) drive 514, and a medium I/F 516.

The CPU 501 controls the overall operation of the computer 500 in accordance with a program. The ROM 502 stores programs such as an initial program loader (IPL) used to boot the CPU 501. The RAM 503 is used as a work area for the CPU 501. The HD 504 stores various types of data such as programs. The HDD controller 505 controls the reading or writing of the various types of data from or to the HD 504 under the control of the CPU 501.

The display 506 displays various pieces of information such as a cursor, a menu, a window, characters, or images. The display 506 may be, for example, a head-mounted display (HMD) or a head-up display (HUD). The external device connection I/F 508 is an interface that connects the computer to various types of external devices. In this case, examples of external devices include, for example, a universal serial bus

(USB) memory and the like.

The network I/F 509 is an interface that allows the computer to perform data communication using the network 20. The data bus 510 may be, for example, an address bus or a data bus that electrically connects the components such as the CPU 501 with each other.

The keyboard 511 is an example of an input device that includes a plurality of keys for inputting characters, numerals, and/or various types of instructions. The pointing device 512 is an example of an input device for selecting or executing various types of instructions, selecting a target for processing, or moving a cursor. The DVD-RW drive 514 controls reading or writing of various types of data from or to a DVD-RW 513, which is an example of a removable storage medium. Note that the removable storage medium is not limited to the DVD-RW, and may be, for example, a digital versatile disk-recordable (DVD-R). The media I/F 516 controls the reading or writing (storing) of data from or to a storage medium 515 such as a flash memory or the like.

Note that the hardware configuration illustrated in FIG. 2 is merely an example. The computer according to the embodiment may have a configuration that does not include some of the components illustrated in FIG. 2 or may have a configuration that includes a component other than the components illustrated in FIG. 2. Since various types of configurations are applicable to cases in which the user terminal 14 illustrated in FIG. 1 is other than a PC, illustrations thereof will be omitted.

<Functional Configuration>

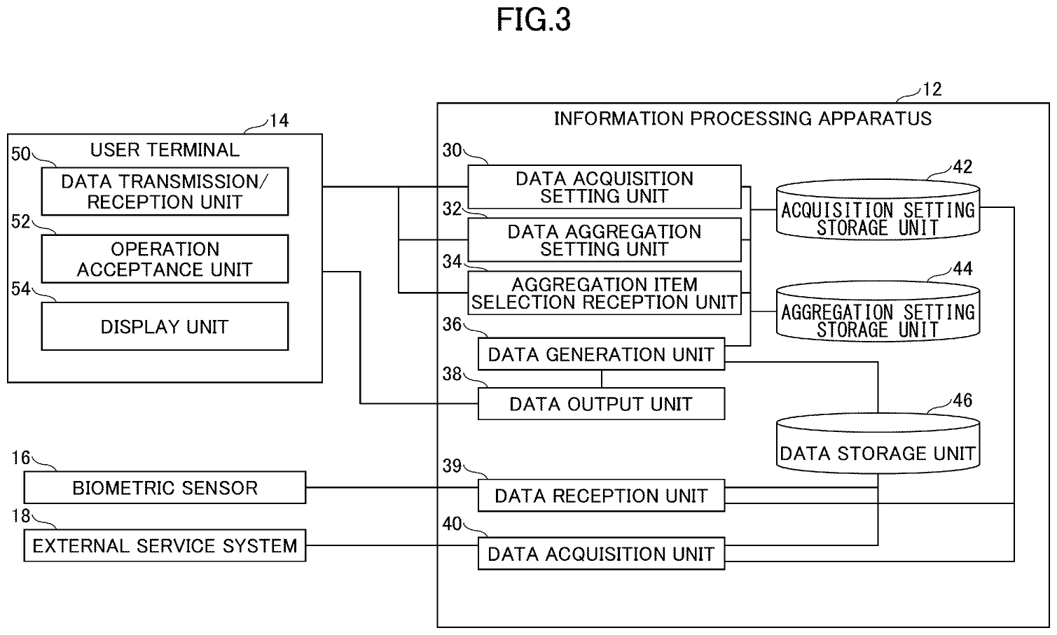

The information processing system 1 according to the embodiment may be implemented by, for example, a functional configuration as illustrated in FIG. 3. FIG. 3 is a block diagram illustrating an example of the functional configuration of the information processing system according to the embodiment. Note that components and/or configurations unnecessary for the description of the embodiment have been accordingly omitted. The information processing apparatus 12, the user terminal 14, the biometric sensor 16, and the external service system 18 each execute programs such as an OS and an application to implement, for example, functions as those that will be described with reference to FIG. 3.

The information processing apparatus 12 illustrated in FIG. 3 includes a data acquisition setting unit 30, a data aggregation setting unit 32, an aggregation item selection reception unit 34, a data generation unit 36, a data output unit 38, a data reception unit 39, a data acquisition unit 40, an acquisition setting storage unit 42, an aggregation setting storage unit 44, and a data storage unit 46.

The data acquisition setting unit 30 receives, from the user operating the user terminal 14, the data acquisition settings for receiving the biometric data from the biometric sensor 16. The data acquisition setting unit 30 also receives, from the user operating the user terminal 14, the data acquisition settings for acquiring the data related to the activity history of the user from the external service system 18. For example, the data acquisition setting unit 30 causes the user terminal 14 to display a data acquisition settings UI, and receives the data acquisition settings from the user.

The data aggregation setting unit 32 receives, from the user operating the user terminal 14, the data aggregation settings for each of one or more aggregation items. The biometric data received from the biometric sensor 16 and the data related to the user's activity history acquired from the external service system 18 in accordance with the data acquisition settings can be aggregated and displayed for each of one or more aggregation items based on the data aggregation settings received from the user. The data aggregation settings include, for each aggregation item, the aggregation setting information in which information to be used for aggregation has been set for the biometric data received from the biometric sensor 16 and the data related to the user's activity history acquired from the external service system 18. For example, the data aggregation setting unit 32 causes the user terminal 14 to display a data aggregation settings UI, and receives the data aggregation settings from the user.

The acquisition setting storage unit 42 stores the data acquisition settings that the data acquisition setting unit 30 received from the user. The aggregation setting storage unit 44 stores the data aggregation settings that the data aggregation setting unit 32 received from the user. In the data aggregation settings, each aggregation item (chart type) that can be used by the user has been defined in accordance with the data acquisition settings. The aggregation item selection reception unit 34 selects, based on the data aggregation settings, the chart types usable by the user in accordance with the data acquisition settings stored in the acquisition setting storage unit 42. The aggregation item selection reception unit 34 also receives, from the user operating the user terminal 14, the selection of the chart type to be output.

The data generation unit 36 generates aggregated data corresponding to the chart type selected by the user. The data storage unit 46 stores the biometric data received from the biometric sensor 16 and the data related to the user's activity history acquired from the external service system 18. The data generation unit 36 generates the aggregated data based on the biometric data and the data related to the user's activity history stored in the data storage unit 46 and the data aggregation settings stored in the aggregation setting storage unit 44. More specifically, the data generation unit 36 generates the aggregated data by visualizing the biometric data and the data related to the user's activity history, which are stored in the data storage unit 46, in a form that enables analysis of trends in time series.

The data output unit 38 outputs the aggregated data generated by the data generation unit 36. The data output unit 38 causes the user terminal 14 to display a UI including the aggregated data that have been visualized in a form that enables analysis of trends in time series. Note that, as described above, the aggregated data are generated by aggregating the biometric data received from the biometric sensor 16 and the data related to the user's activity history acquired from the external service system 18 that are stored in the data storage unit 46.

The data reception unit 39 receives the biometric data from the biometric sensor 16 in accordance with the data acquisition settings stored in the acquisition setting storage unit 42. The data acquisition unit 40 acquires the data related to the user's activity history from the external service system 18 in accordance with the data acquisition settings stored in the acquisition setting storage unit 42. The data storage unit 46 stores the biometric data, which the data reception unit 39 receives from the biometric sensor 16, and the data related to the user's activity history, which the data acquisition unit 40 acquires from the external service system 18.

The user terminal 14 illustrated in FIG. 3 includes a data transmission/reception unit 50, an operation acceptance unit 52, and a display unit 54. The data transmission/reception unit 50 executes data communication with the information processing apparatus 12. The operation acceptance unit 52 accepts operation from the user and executes various types of processing corresponding to the operation. The display unit 54 displays, for example, an UI provided from the information processing apparatus 12.

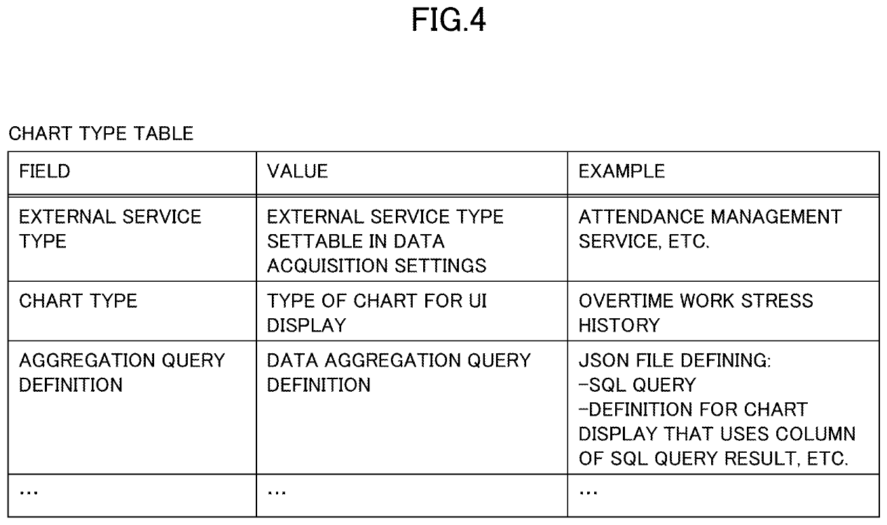

The aggregation setting storage unit 44 stores, as an example of aggregation settings information, a chart type table illustrated in FIG. 4. FIG. 4 is a view illustrating an example of the configuration of a chart type table. Note that a chart type table that has been defined in any of the information processing apparatuses 12 in the aggregation system 10 may be used as the chart type table.

The chart type table illustrated in FIG. 4 includes, for example, the external service type, the chart type, and the aggregation query definition. The external service type represents the types of external services settable in the data acquisition settings. Examples of external service types include an attendance management service and the like.

The chart type represents the type of chart (the name of the chart) to be displayed on the UI. For example, overtime work stress history and the like are defined as chart types. The aggregation query definition represents the definition of the data aggregation query. The aggregation query is defined by, for example, a JSON file in which an SQL query, a definition for chart display using the column of an SQL query result, or the like has been defined. An SQL query is a command written in accordance with the usage of SQL, which is an example of a language for operating a database. Note that SQL is merely an example, and other languages for database operation may be used. The definition for chart display using the column of an SQL query result is information used to display a chart from the data acquired from the data storage unit 46 based on the SQL query result. A JSON file is an example of a data file that is in a text-based format.

<Processing>

The processing performed after the user using the aggregation system 10 according to the embodiment has configured the data acquisition settings on the data acquisition settings UI of the aggregation system 10 will be described hereinafter. The data acquisition settings received from the user are stored in the acquisition setting storage unit 42. The data acquisition settings stored in the acquisition setting storage unit 42 include, for example, the external service type, the access destination of the external service such as a uniform resource locator (URL) of an application programming interface (API) for data acquisition, and the parameters for accessing the access destination. The data acquisition settings stored in the acquisition setting storage unit 42 also include, for example, information used to receive the biometric data from the biometric sensor 16. For example, the information used to receive the biometric data from the biometric sensor 16 includes the type of the biometric sensor 16, the authentication information, the communication method, the access destination of the biometric sensor 16, the parameters for accessing the access destination, and the like.

Furthermore, the processing performed after the user using the aggregation system 10 according to the embodiment has configured the data aggregation settings on the data aggregation settings UI of the aggregation system 10 will also be described hereinafter. The aggregation setting storage unit 44 stores the data aggregation settings received from the user.

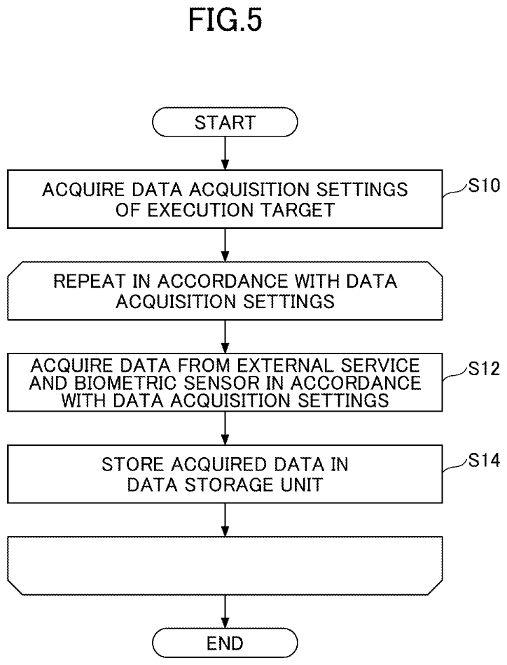

The data reception unit 39 of the information processing apparatus 12 receives biometric data from the biometric sensor 16 based on the data acquisition settings, which are stored in the acquisition setting storage unit 42, in accordance with, for example, the procedure as illustrated in FIG. 5. The data acquisition unit 40 also acquires the data related to the user's activity history from the external service system 18 based on the data acquisition settings, which are stored in the acquisition setting storage unit 42, in accordance with, for example, the procedure illustrated in FIG. 5. FIG. 5 is a flowchart illustrating an example of data acquisition processing.

In step S10, the data acquisition settings to be executed are acquired from the acquisition setting storage unit 42 by the data reception unit 39 and the data acquisition unit 40. The data acquisition settings to be executed are the data acquisition settings at the timing of data acquisition. The timing of data acquisition may be the timing at which a predetermined period has elapsed, the timing at which a request for chart display is received from the user, or the like.

The processes of steps S12 and S14 are repeated in accordance with the number of data acquisition settings that are read out from the acquisition setting storage unit 42 by the data reception unit 39 and the data acquisition unit 40. In step S12, the data reception unit 39 receives the biometric data from the biometric sensor 16 in accordance with the data acquisition settings acquired from the acquisition setting storage unit 42. The data acquisition unit 40 acquires the data related to the user's activity history from the external service system 18 in accordance with the data acquisition settings acquired from the acquisition setting storage unit 42. In step S14, the data reception unit 39 stores the biometric data received from the biometric sensor 16 in the data storage unit 46. The data acquisition unit 40 stores the data related to the user's activity history acquired from the external service system 18 in the data storage unit 46. The data storage unit 46 may be, for example, a database, a data warehouse, or a data lake.

According to the processing illustrated in the flowchart of FIG. 5, accepting data acquisition settings from the user allows the biometric sensor 16 and the external service system 18 from which data are to be acquired to be changed, thus enabling data acquisition from variety of biometric sensors 16 and external service systems 18.

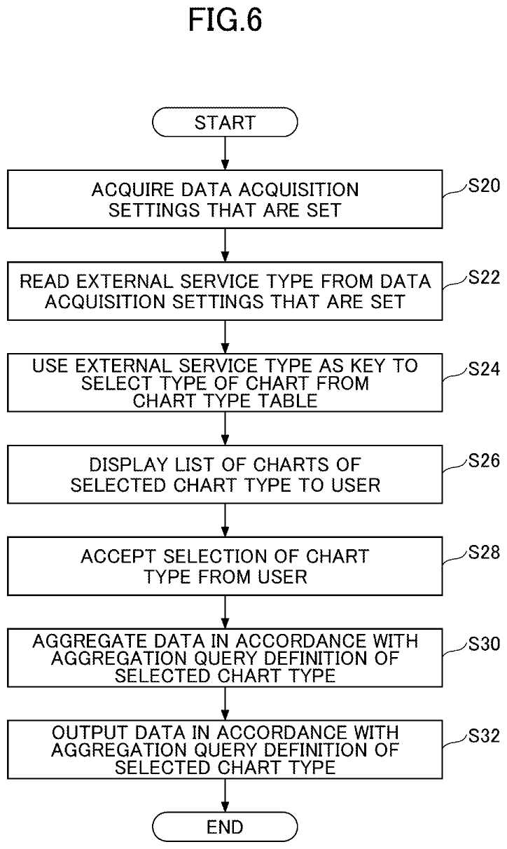

Based on the data acquisition settings stored in the acquisition setting storage unit 42 and the data aggregation settings stored in the aggregation setting storage unit 44, the aggregation item selection reception unit 34, the data generation unit 36, and the data output unit 38 of the information processing apparatus 12 output the aggregated data of the data stored in the data storage unit 46 in accordance with, for example, the procedure illustrated in FIG. 6.

FIG. 6 is a flowchart illustrating an example of data output processing. In step S20, the aggregation item selection reception unit 34 acquires the data acquisition settings that have been set by the user from the acquisition setting storage unit 42. In step S22, the aggregation item selection reception unit 34 reads the external service type from the data acquisition settings that have been set. The external service type that is read in step S22 is the data related to the user's activity history that can be acquired from the external service system 18 based on the data acquisition settings that have been set.

In step S24, the aggregation item selection reception unit 34 selects the chart type from the chart type table illustrated in FIG. 4 by using, as a key, the external service type read in step S22. The chart type selected in step S24 becomes the list of charts that can be output by the user. In step S26, the aggregation item selection reception unit 34 causes the user terminal 14 to display the list of charts representing the chart type selected in step S24.

In step S28, the aggregation item selection reception unit 34 receives the user's chart type selection in response to the user's operation on the user terminal 14. In step S30, the data generation unit 36 reads, from the chart type table illustrated in FIG. 4, the aggregation query definition of the chart type selected by the user in step S28, and executes data aggregation in accordance with the aggregation query definition.

Based on, for example, the SQL query read as the aggregation query definition from the chart type table illustrated in FIG. 4, the data generation unit 36 executes data aggregation by acquiring the biometric data received from the biometric sensor 16 and the data related to the user's activity history acquired from the external service system 18 from the data storage unit 46.

In step S32, the data output unit 38 outputs data in accordance with the aggregation query definition of the user's chart type selection received in step S28. For example, based on the definition for chart display using the column of an SQL query result that is read as the aggregation query definition from the chart type table illustrated in FIG. 4, the data output unit 38 can cause the user terminal 14 to display a UI that includes a chart visualizing the result of data aggregation executed in step S30.

Note that the data output from the aggregation system 10 to the user terminal 14 may be performed so that a chart is displayed on a dashboard or may be performed so that a report file is generated and transmitted via email. A dashboard is, for example, a screen that displays the result of data acquisition from multiple information sources and processing on the acquired data.

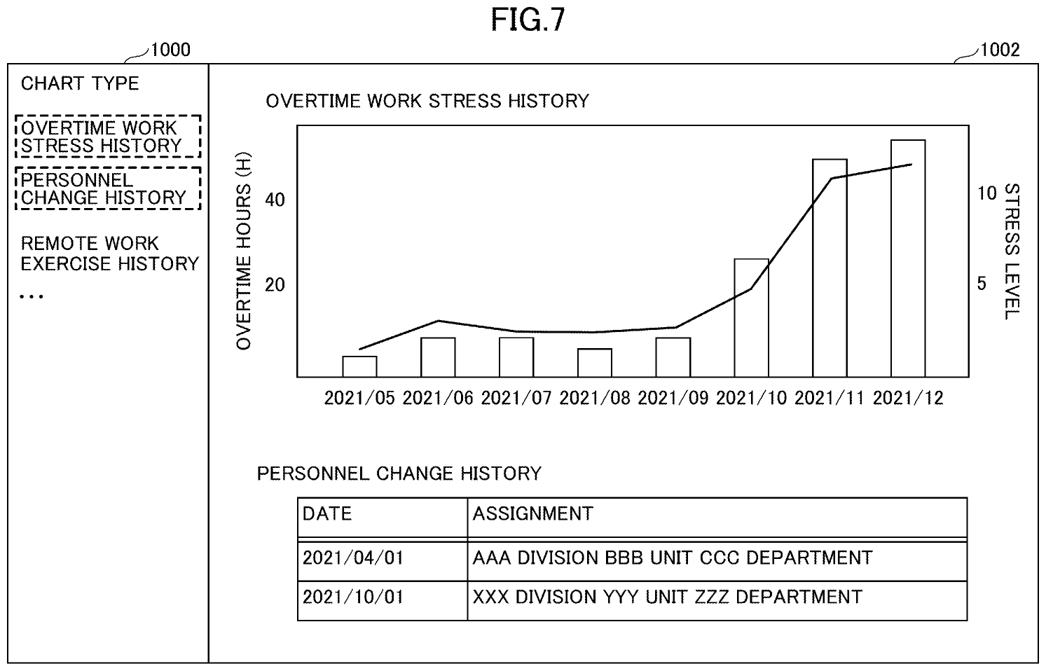

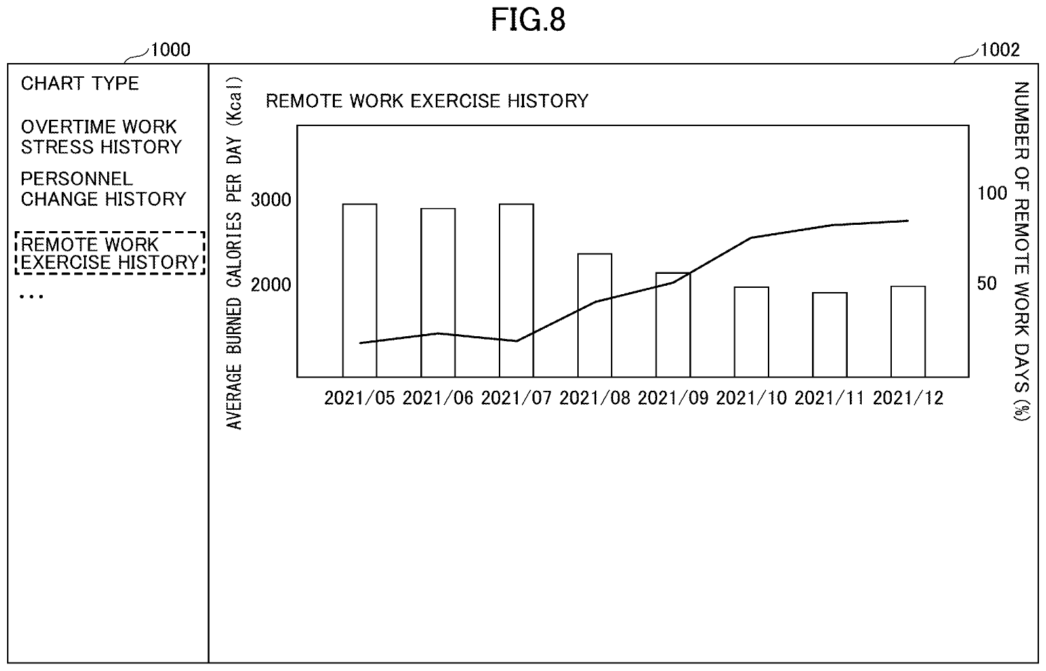

The data output unit 38 causes the user terminal 14 to display a UI screen that includes charts as illustrated in FIGS. 7 and 8. FIGS. 7 and 8 are views each illustrating an example of a UI screen including charts. FIGS. 7 and 8 illustrate examples of UI screens that allow the user to select each chart they want to use.

In each of the screens illustrated in FIGS. 7 and 8, the display area is divided into two columns (panes). A column 1000 on the left side is a chart type menu column. A column 1002 on the right side is a chart display column. The chart type menu column displays a list of chart types that can be selected by the user operating the user terminal 14. The user operating the user terminal 14 selects the chart type they want to display on the chart display column from the chart type menu column.

For example, in FIG. 7, an overtime work stress history, a personnel change history, and a remote work exercise history are displayed as chart types in the chart type menu column. In the example illustrated in FIG. 7, a user (for example, User A) operating the user terminal 14 has selected the overtime work stress history and the personnel change history in the chart type menu column. Since the user operating the user terminal 14 has selected the overtime work stress history and the personnel change history in the chart type menu column, the respective charts for the overtime work stress history and the personnel change history are displayed in the chart display column.

The stress level received from a user's (for example, User B) biometric sensor 16 and the overtime hours acquired from one external service system 18, which is an attendance management service, have been processed into aggregated data visualized in a form that enables analysis of trends in time series, and a chart corresponding to the aggregated data is displayed on the screen illustrated in FIG. 7. The personnel change history acquired from another external service 18, which is an organization management service, is also displayed as a chart on the screen illustrated in FIG. 7.

The screen displaying these two types of charts in FIG. 7 visualizes the data in a form that enables analysis of medium- to long-term trends. Hence, for example, User A that refers to the screen illustrated in FIG. 7 can analyze that the stress level of User B is increasing in accordance with the increase in the overtime hours of User B, and that this increase in the stress level is very likely due to a change in work operations brought on by an organizational transfer. Using the screen illustrated in FIG. 7 allows User A to consider changing the personnel assignment to reduce the stress level of User B. Furthermore, the aggregation system 10 according to the embodiment can also use the information displayed on the screen in FIG. 7 to alert a supervisor or an occupational physician of the information about User B whose stress level is high.

FIG. 8 illustrates an example in which a user operating the user terminal 14 has selected the remote work exercise history from the chart type menu column. Since the user operating the user terminal 14 has selected the remote work exercise history from the chart type menu column, the chart illustrating the remote work exercise history is displayed on the chart display column.

The average burned calories per day received from a user's biometric sensor 16 and the number of remote work days acquired from the external service system 18, which is an attendance management service, have been processed into aggregated data visualized in a form that enables analysis of trends in time series, and a chart corresponding to the aggregated data is displayed on the screen illustrated in FIG. 8.

The screen displaying such a chart in FIG. 8 visualizes the data in a form that enables analysis of medium- to long-term trends. Hence, for example, a user who refers to the screen illustrated in FIG. 8 can analyze that there is a tendency toward a lack of exercise due to remote work. Further, by using the information displayed on the screen illustrated in FIG. 8, the aggregation system 10 according to the embodiment is able to, for example, automatically detect a user who has a high tendency to lack exercise due to remote work, and transmit health advice to the detected user.

Note that the chart types displayed in the chart type menu and the number of charts to be displayed on the chart display column need not be in a one-to-one relationship. For example, a plurality of charts may be displayed in the chart display column when a single chart type is selected from the chart types displayed on the chart type menu column.

According to the first embodiment, aggregated data desired by the user can be provided based on the data acquired from the biometric sensor 16 and the external service system(s) 18.

[Second Embodiment]

In the first embodiment, the aggregation system 10 received the biometric data from the biometric sensor 16. The second embodiment describes an example in which the aggregation system 10 acquires the biometric data from the external service system 18 that has received the biometric data from the biometric sensor 16 and is managing the received biometric data.

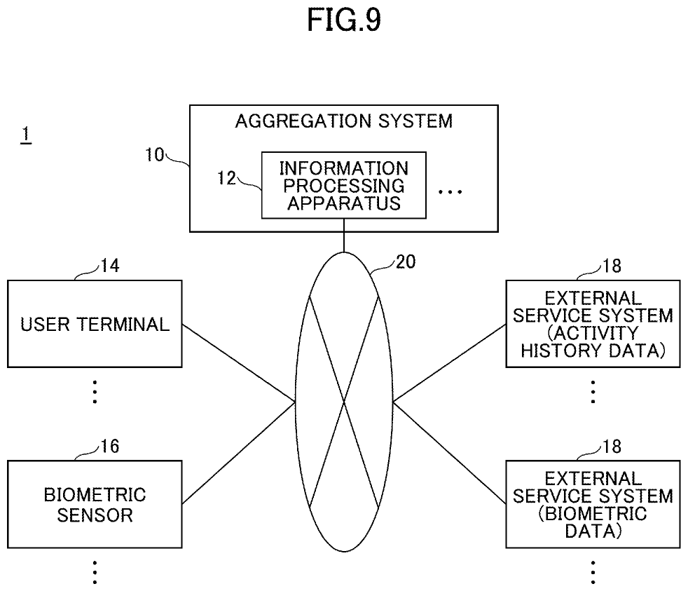

Note that, excluding some parts, the second embodiment is similar to the first embodiment. Thus, a description thereof will be suitably omitted. FIG. 9 is a diagram illustrating an example of the configuration of an information processing system according to the embodiment. The information processing system 1 illustrated in FIG. 9 differs from the information processing system 1 illustrated in FIG. 1 in that the external service system 18 that manages the activity history data and the external service system 18 that manages the biometric data are present.

The information processing apparatus 12 executes processing to cause the user terminal 14 to display aggregated data based on the data (activity history data) related to a user's activity history, which is acquired from the external service system 18 managing the activity history data, and the data (biometric data) related to the user's health, which is acquired from the external service system 18 managing the biometric data.

The external service system 18 managing the biometric data receives and manages the biometric data measured by the biometric sensor 16. The external service system 18 managing the biometric data provides, for example, a service that processes, analyzes, and visualizes the user's biometric data that is being managed, and causes the resulting data to be displayed on the user terminal 14.

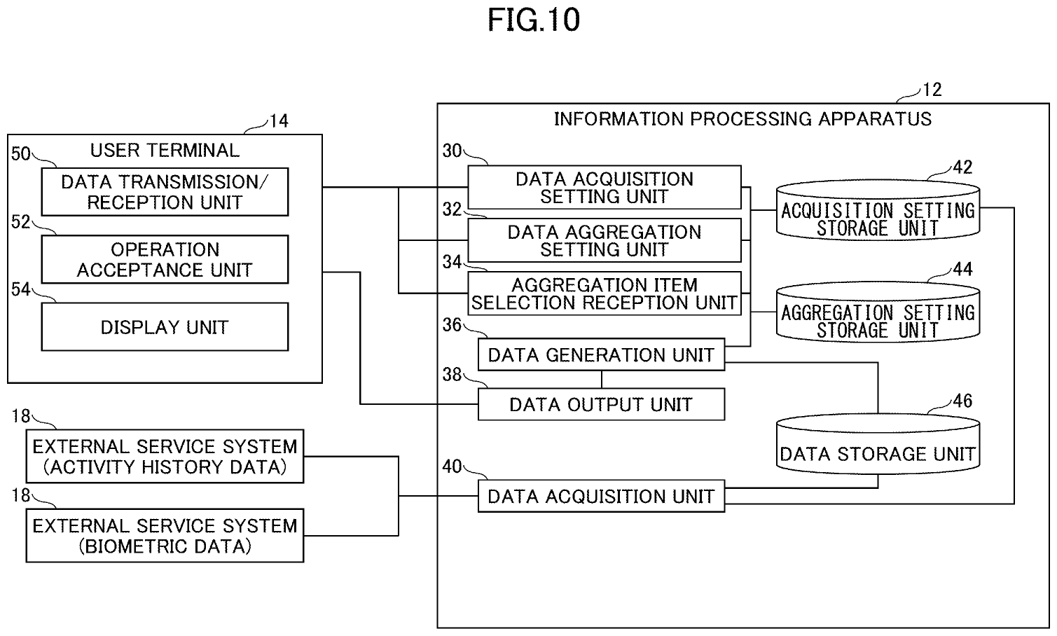

The information processing system 1 according to the second embodiment may be implemented by, for example, a functional configuration as illustrated in FIG. 10. FIG. 10 is a block diagram illustrating an example of the functional configuration of the information processing system according to the embodiment. As the functional configuration illustrated in FIG. 10 is mostly similar to the functional configuration illustrated in FIG. 3 excluding some parts, a description thereof will be suitably omitted.

The data acquisition setting unit 30 of the information processing apparatus 12 illustrated in FIG. 10 receives, from the user operating the user terminal 14, the data acquisition settings for acquiring the biometric data of the biometric sensor 16 from the external service system 18. The data acquisition setting unit 30 also receives, from the user operating the user terminal 14, the data acquisition settings for acquiring a user's activity history data from the external service system 18.

The data aggregation setting unit 32 receives, from the user operating the user terminal 14, the data aggregation settings for aggregating the biometric data and the activity history data acquired from the external service systems 18 in accordance with the data acquisition settings.

The data output unit 38 outputs the aggregated data generated by the data generation unit 36. The data output unit 38 causes the user terminal 14 to display a UI that includes the aggregated data visualizing the biometric data and the activity history data, which are stored in the data storage unit 46, in a form that enables analysis of trends in time series.

The data acquisition unit 40 acquires the biometric data and the activity history data from the corresponding external service systems 18 in accordance with the data acquisition settings stored in the acquisition setting storage unit 42. The data storage unit 46 stores the biometric data and the activity history data acquired by the data acquisition unit 40. As other aspects of the functional configuration are the same as those of the first embodiment, a description thereof will be omitted.

According to the second embodiment, aggregated data desired by a user can be provided based on a user's activity history data and the biometric data that are acquired from the corresponding external service systems 18.

Each of the functions described in the above embodiments can be implemented by one or more processing circuits (circuitry). A processing circuit or circuitry may include a processor programmed to use software to execute a function in a similar manner to a processor implemented by an electronic circuit. A processing circuit or circuitry may also include devices such as an application specific integrated circuit (ASIC), a digital signal processor (DSP), a field programmable gate array (FPGA), conventional circuit components, and the like that are designed to execute each function as described above.

The group of apparatuses exemplified above merely illustrate a single example of a plurality of computing environments in which the disclosed embodiments can be implemented. The present disclosure is not limited to the disclosed embodiments, and the components of the embodiments include components that can be easily imagined by a person skilled in the art, substantially same components, and/or equivalent components. Furthermore, various omissions, substitutions, changes, and combinations can be made with respect to the components without departing from the scope of the disclosure or the claims.

What is claimed is:

1. An information processing apparatus comprising:
a memory; and
circuitry configured to
connect to an external service that manages activity history of a user,
acquire, from the external service, first data related to the activity history of the user,
receive second data related to the user's health that is measured by a biometric sensor,
store, in the memory, aggregation setting information in which information used for aggregation of the first data and the second data is set for one or more aggregation items, for each of which the first data and the second data are aggregated and displayed,
generate, based on the acquired first data, the received second data, and the stored aggregation setting information, aggregated data corresponding to at least one aggregation item that is selected by the user from the one or more aggregation items, and
output the generated aggregated data corresponding to the at least one aggregation item that is selected by the user.

2. The information processing apparatus of claim 1, wherein the circuitry is further configured to output the aggregated data in which the first data and the second data are visualized in a form that enables analysis of a trend in time series.

3. The information processing apparatus of claim 1, wherein the circuitry is further configured to acquire the second data from the external service.

4. The information processing apparatus of claim 1, wherein the circuitry is further configured to store the acquired first data and the received second data in the memory.

5. The information processing apparatus of claim 1, wherein the circuitry is further configured to acquire the first data in accordance with settings of a type of the external service, an access destination of the external service, and a parameter used for accessing the access destination.

6. The information processing apparatus of claim 1, wherein the circuitry is further configured to accept the at least one aggregation item that is selected by the user from the one or more aggregation items.

7. The information processing apparatus of claim 1, wherein the second data includes data acquired from the biometric sensor of a wearable device worn by the user.

8. An information processing system comprising:
an information processing apparatus including a memory and first circuitry; and
a user terminal including second circuitry,
the user terminal and the information processing apparatus being communicably connected to one another,
the first circuitry being configured to
connect to an external service that manages activity history of a user,
acquire, from the external service, first data related to the activity history of the user,
receive second data related to the user's health that is measured by a biometric sensor,
store, in the memory, aggregation setting information in which information used for aggregation of the first data and the second data is set for one or more aggregation items, for each of which the first data and the second data are aggregated and displayed,
generate, based on the acquired first data, the received second data, and the stored aggregation setting information, aggregated data corresponding to at least one aggregation item that is selected by the user from the one or more aggregation items, and
output the generated aggregated data corresponding to the at least one aggregation item that is selected by the user, and
the second circuitry being configured to
display the aggregated data output from the information processing apparatus.

9. An information processing method executed by an information processing apparatus, the information processing method comprising:
connecting to an external service that manages activity history of a user;
acquiring, from the external service, first data related to the activity history of the user;
receiving second data related to the user's health that is measured by a biometric sensor;
storing, in a memory, aggregation setting information in which information used for aggregation of the first data and the second data is set for one or more aggregation items, for each of which the first data and the second data are aggregated and displayed;
generating, based on the acquired first data, the received second data, and the stored aggregation setting information, aggregated data corresponding to at least one aggregation item that is selected by the user from the one or more aggregation items; and
outputting the generated aggregated data corresponding to the at least one aggregation item that is selected by the user.
